(19) United States
(12) Patent Application Publication
(10) Pub. No.: US 2024/0341753 A1
(43) Pub. Date: Oct. 17, 2024

(54) BIO-DISSOLVABLE SUTURING NEEDLE

(71) Applicants: Brian Gilchrist, Brooklyn, NY (US); Pratibha Vemulapalli, New York, NY (US)

(72) Inventors: Brian Gilchrist, Brooklyn, NY (US); Pratibha Vemulapalli, New York, NY (US)

(21) Appl. No.: 18/634,059

(22) Filed: Apr. 12, 2024

Related U.S. Application Data
(60) Provisional application No. 63/495,597, filed on Apr. 12, 2023.

(51) Int. Cl. A61B 17/06; A61B 17/00

(52) U.S. Cl. CPC A61B 17/06066; A61B 2017/00004; A61B 2017/00526; A61B 2017/06071; A61B 2017/0608

(57) ABSTRACT

A suturing needle which is dissolvable if left within subcutaneous tissue or within a human body cavity (i.e., bio-dissolvable, bioabsorbable, or bioresorbable). A bio-dissolvable suturing needle can have a tip and a base, and the base can be connected to suturing thread, such that if the bio-dissolvable suturing needle is inadvertently left in a patient, the needle will dissolve without the need to perform an additional surgery or operation on the patient.

### CROSS-REFERENCE

This application claims priority to U.S. provisional patent application No. 63/495,597, filed Apr. 12, 2023, the contents of which are incorporated herein in their entirety.

### BACKGROUND

### 1. Technical Field

The present disclosure relates to suturing needles, and more specifically to a suturing needle which is dissolvable if left within subcutaneous tissue or within a human body cavity (i.e., bio-dissolvable).

### 2. Introduction

Needles left inside a patient at the time of surgery can result in extra hospital time, and can result in extra anesthesia, possible reoperations, and/or malpractice implications for the hospitals and surgeons, with patients being imperiled both physically and mentally.

### SUMMARY

Additional features and advantages of the disclosure will be set forth in the description that follows, and in part will be understood from the description, or can be learned by practice of the herein disclosed principles. The features and advantages of the disclosure can be realized and obtained by means of the instruments and combinations particularly pointed out in the appended claims. These and other features of the disclosure will become more fully apparent from the following description and appended claims, or can be learned by the practice of the principles set forth herein.

Disclosed are a bio-dissolvable suturing needle and methods of manufacturing the bio-dissolvable suturing needle.

An exemplary bio-dissolvable suturing needle formed as disclosed herein can include: a tip, and a base.

An exemplary method of making a bio-dissolvable suture needle can include the steps of: injecting a bio-dissolvable material into a mold having a suture needle form, resulting in a bio-dissolvable suture needle; and cooling the mold until the bio-dissolvable suture needle solidifies.

Another exemplary method of making a bio-dissolvable suture needle can include the steps of: retrieving, from a database, a three dimensional (3D) suture needle design; uploading the three dimensional suture needle design to a 3D printer; providing bio-dissolvable material to the 3D printer; and forming, via the 3D printer, the bio-dissolvable material into the bio-dissolvable suture needle according to the 3D suture needle design.

### BRIEF DESCRIPTION OF THE DRAWINGS

FIG. 1 illustrates an example of a bio-dissolvable needle with suture material;

FIG. 2 illustrates an example of a mold for multiple conjoined bio-dissolvable needles; and

FIG. 3 illustrates an example method embodiment.

### DETAILED DESCRIPTION

Various embodiments of the disclosure are described in detail below. While specific implementations are described, this is done for illustration purposes only. Other components and configurations may be used without parting from the spirit and scope of the disclosure.

Needles left inside a patient at the time of surgery should never happen. Nevertheless, mistakes happen, with common errors including: miscounts by nurses (where the nurses are counting needles used and retrieved, and erroneously believe the needles have been removed), the needles ricocheting out of small incisions (or trocars), resulting in the needles becoming “lost in the abdominal cavity; and emergency conversions or trauma surgery, where quick action and quick counting of used/retrieved needles results in erroneous counting and tracking of needles used.

When it is recognized that needles are in a patient (via x-ray, or an identification based on the “counts” (i.e., the number of needles used)) extra time and extra anesthesia can be required, often resulting in longer hospital stays and possible reoperations to retrieve the needle. This can also lead to mental and/or physical problems for the patient. Retained needles can also have negative malpractice implications for hospitals and surgeons.

By contrast, suturing needles which are manufactured according to the principles disclosed herein can dissolve if retained in the human body, such that reoperations are unnecessary. Kindly note that while both bioabsorbable and bioresorbable materials are available, bio-dissolvable as used herein refers to the ability for the patient's body to completely absorb the needle should it be left behind.

Needles manufactured as disclosed herein can be made of the same absorbable tack material used for hernia mesh tacks (e.g., PLA (polylactic acid an absorbable reusable thermoplastic polyester copolymer) or the materials used in modern stents (e.g., PLGA (Poly Lactic-co-glycolic Acid) polymer, or Magnesium) which are biodegradable polymers that are physically strong and will dissolve with time. During use, most needles used in the abdominal cavity are used only 1-2 times (aka “throws”) so “dullness” of the bio-dissolvable needs will not be a factor during usage. Instead, the bio-dissolvable needles disclosed herein only need to retain durability and strength for 1-2 throws and/or 1-2 grasps with needle drivers and graspers.

Exemplary benefits of bio-dissolvable needles can include: Avoidance of foreign body within a patient; avoidance of unnecessary needle sticks to healthcare providers; decreased time to complete operation (patient anesthetized) with no needle counts; the ability of OR (Operating Room) team to focus on other items to count that are not bio-dissolvable (e.g., retractors, sponges), and/or decreased anesthetic time for a patient when needles are lost.

Safety Profile. PGLA and PLA polymers has already been proven as a viable absorbable material, with absorbable mesh tacks used in hernia repair. Whereas absorbable mesh tacks for hernias may dissolve over a period of ten or more years in the body, needles simply must be shelf stable until use, then they can dissolve almost immediately within patients. Most PGLA/PLA materials can dissolve within 20-30 days without gamma radiation (depending on conditions), making PGLA and PLA an ideal material for forming needles, as needles (unlike tacks) do not need to hold anything in place for 6-8 weeks during a healing process. Non-limiting, exemplary types of needles can include robotic and laparoscopic bowel anastomotic intra-abdominal suturing needles, which can be paired with sutures formed of the same material (or another material). Suture materials needs do not need to change-only needle manufacturing such that the needle is formed with a bio-dissolvable alloy. As a non-limiting example of a bio-dissolvable alloy, the alloy can be a MAX-PRENE® polymer by POLY-MED, INC., with MAX-PRENE® using glycolide/lactide linear random copolymers.

Properties of a Suturing Needle. The following properties apply to suturing needles, both bio-dissolvable as formed according to the principles disclosed herein and non-dissolvable.

A) Tapered—sharp and pointed to go through tissue with minimal resistance but does not need a blade.

B) Slim—as slim as possible without compromising strength. Slim so that the needle caliber does not exceed the suture caliber and leave gaps.

C) Rigid—rigid enough to resist bending for 3-4 throws through tissue. Breaking during surgery is not ideal but is tolerable.

D) Stable in the needle holder (both open, robotic, laparoscopic and micro needle holders)

E) Sterile to prevent micro-organisms or foreign material into the wound.

Manufacturing. Manufacturing of bio-dissolvable suture needles of all sizes is within the scope of this disclosure, but primarily intra-abdominal needles could be manufactured through conventional manufacturing techniques to produce tapered needles. The manufacturing process can include one or more of the processes described below, or other manufacturing processes known to those of skill in the art. The needle manufacturing process can result in the bio-dissolvable suture needle have a tapered needle with a blunt tip, or a needle with a cutting tip. Needles can have a sharpness (also known as a “rise distance”) sufficient to go through tissue, and can have an equivalent sharpness to non-bio-dissolvable needles currently available. The bio-dissolvable suture needle can, in some configurations, have a needle hole or attachment point for attaching suture materials, such as (but not limited to), VICRYL, polypropylene or polydioxanone. Additional non-limiting exemplary materials which can be used to form the bio-dissolvable suture needle and/or bio-dissolvable suture material are listed below.

In other configurations, because the bio-dissolvable suture needle and the bio-dissolvable suture material can be made of the same material, in some configurations the manufacturing process can have the bio-dissolvable suture material as a continuous extension of the bio-dissolvable suture needle. In other words, the bio-dissolvable suture needle can be manufactured so that bio-dissolvable “thread” (the suture material) comes continuously attached to the bio-dissolvable suture needle. Such configurations would not have the suture material welded to the needle, resulting in a cleaner and easier suture passage. Therefore, no crimping necessary providing atraumatic attachment with suture that is the same diameter as needle hole in tissue.

Manufactured bio-dissolvable suture needles can be formed into different needle shapes, including curved needles or straight needles. The curved needles can include all types of needles for different parts of the body and procedures. Non-limiting examples of curved needles can include: ½ circle, ⅜ circle, ¼ circle, ⅝ circle, J shape, and a ½ curve.

### Exemplary Manufacturing Process: 3-D Printing

In this exemplary process, the manufacturer uses a 3-D (3 Dimensional) CAD (Computer Aided Design) file which has the dimensions and specific parameters of the suture needle. The bio-dissolvable/biodegradable material being used to form the bio-dissolvable suture needles must be in a form which allows the printer to form the needle. Typically, this is a filament form, meaning a spool of polymer/thermoplastics which, when heated, can be molded into whatever form is dictated by the 3-D printer. In this example, the 3-D printer would heat the material into the shape of one or more needles and the suture, which could then be used by the surgeon as needed.

### Exemplary Manufacturing Process: Molds

Using Injection molds to force the bio-dissolvable materials into the shape of a needle. The bio-dissolvable materials are first heated to make them malleable, then the malleable material is placed within the molds, forcing the materials to take the shape of the mold. The mold is cooled until it is hard and in the shape of the needle. The material for the mold can be stainless steel and/or any other material known to those of skill in the art.

### Exemplary Manufacturing Process: Hybrid of 3-D Printing and Molds

In this example, a 3-D printer can be used to create a mold, then the bio-dissolvable materials can be placed within the molds to form the needle. In some configurations, the types of materials used to form the molds and the needles can vary, such that the temperature required for manipulating the bio-dissolvable material into needles is different than the melting temperature of the molds.

Non-limiting Examples of Bio-dissolvable Materials. The following non-limiting examples of bio-dissolvable materials can be used to form the bio-dissolvable suture needle and/or the suture materials which attach to the bio-dissolvable suture needles.
PLA (Polylactic Acid)
PGLA
PLGA
PGLA gamma irradiated
Polyglycolic Acid (PGA)
Polydioxanone
Poliglecaprone 25
Biodegradable Magnesium Alloy, commonly used in stents
Alloy AE21 (2% aluminum, 1% rare earths)
Uncoated Alloy AZ31, commonly used in coronary stents
Uncoated WE43 Alloy

Sterilization: Bio-dissolvable needles manufactured as disclosed as disclosed herein (with or without suture material) can be sterilized by using EO (ethylene oxide) sterilization. For example, needles (with or without suture material) can be placed in EO sterilizer pouches, which are commercially available. The EO sterilizer pouches can be placed in an EO gas cannister sterilizer to be sterilized. The EO permeates the pouches and packing material that the needles maybe placed in, resulting in sterilized needles and/or suture material.

Other sterilization methods can include gamma radiation, which can sterilize with equal efficaciousness.

In some configurations, two suture needles can be formed with a common supporting structure. That supporting structure can be part of the manufacturing process and will be cut off. Each suture needle has a tip and a base. The base can be detached from the supporting structure (e.g., by pulling or by twisting the needle with respect to the support structure), such that an individual needle is available for sterilization and/or use.

The needle tip can have various shapes and not limited to circle (also known as “tapered”) and triangle (also known as “cutting”). Tapering down from the tip along the body of the needle to the base, the diameter of the needle can increase. Non-limiting examples of the cross-sectional shape of the needle body can include: a circle, a triangle, a square, a rectangle, a rhombus, a kite, a pentagon, a hexagon, an octagon, or any other shape.

In some configurations the bio-dissolvable suture needles can include a contiguous suturing thread (i.e., the suturing thread is part of the needle). In other configurations, the base of one of the manufactured needles can have suture material fixed to the needle via an eyehole or other mechanical means.

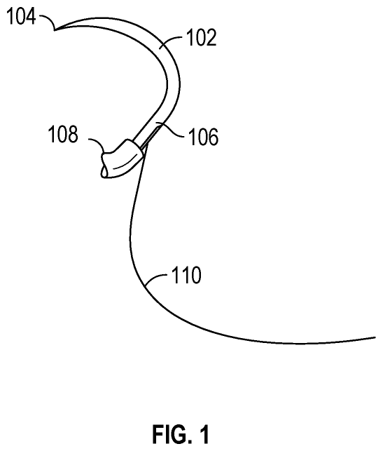

FIG. 1 illustrates an example of a bio-dissolvable needle 102 with suture material 110. As illustrated, the bio-dissolvable needle 102 has a tip (aka a needle point) and a swaged end 106. The suture material 110 can connect to the bio-dissolvable needle 102 through an eye in the swaged end 106. In other configurations, the bio-dissolvable needle 102 can be contiguous with the suture material 110, such that the suture material is a continuation of the bio-dissolvable needle 102.

In configurations where the bio-dissolvable needle 102 has been formed using a mold or a 3D printer, the bio-dissolvable needle 102 may be formed conjoined with other bio-dissolvable needles. In such configurations, the bio-dissolvable needle 102 may need to be twisted or otherwise separated from the other needles. This is illustrated in FIG. 1 by the remaining portion 108, which represents where the bio-dissolvable needle 102 was separated from the other bio-dissolvable needles.

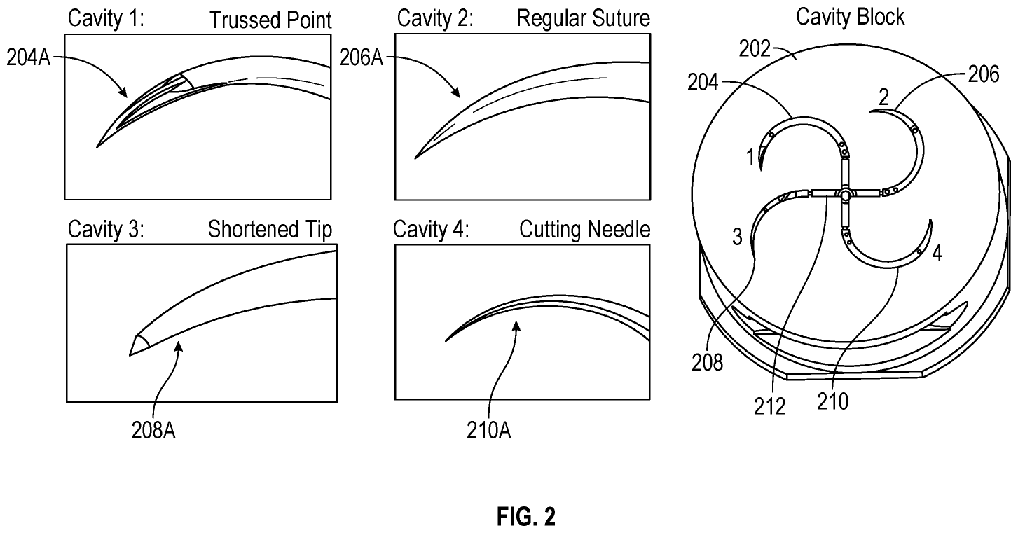

FIG. 2 illustrates an example of a mold 202 for multiple conjoined bio-dissolvable needles 204, 206, 208, 210. The mold 202 can be made of any material known to those of skill in the art. As illustrated, four needles 204, 206, 208, 210 are made simultaneously using the mold 202, with the four needles 204, 206, 208, 210 conjoined by a common base 212 from which the individual needles 204, 206, 208, 210 can be detached (e.g., through twisting, pinching, breaking off, etc.).

In this example, the four needles 204, 206, 208, 210 have distinct types of tips, with enlarged (i.e.., zoomed in) versions illustrated in FIG. 2 by needles 204A, 206A, 208A, and 210A. As illustrated, bio-dissolvable needle 204 is a trussed point, illustrated by 204A. Bio-dissolvable needle 206 is a regular suture, illustrated by 206A. Bio-dissolvable needle 208 has a shortened tip, illustrated by 208A. Bio-dissolvable needle 210 is a cutting needle, illustrated by 210A.

While in FIG. 2 all the needles 204, 206, 208, 210 have distinct types of tips, in other configurations all needles created by a common mold 202 can be identical, whereas in yet other configurations the needles created by a common mold 202 can have multiple needles having a common type and some needles which are individual (e.g., two regular sutures, a cutting needle, and a shortened tip). In addition, while the illustrated example shows four needles 204, 206, 208, 210, in other configurations the number of needles created in a common mold, or in a common batch by a 3D printer, can vary. For example, the number of needles created together may be two, three, four, five, and so on. In yet other configurations, a single needle may be created.

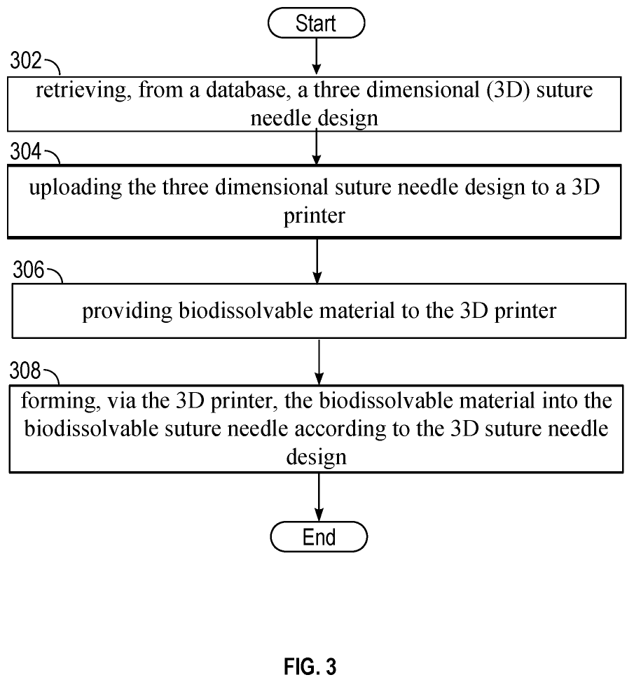

FIG. 3 illustrates an example method embodiment. As illustrated, the method can include: retrieving, from a database, a three dimensional (3D) suture needle design (302); uploading the three dimensional suture needle design to a 3D printer (304); providing bio-dissolvable material to the 3D printer (306); and forming, via the 3D printer, the bio-dissolvable material into the bio-dissolvable suture needle according to the 3D suture needle design (308).

In some configurations, the bio-dissolvable suture needle can be at least one of: a trussed point needle; a regular suture needle; a shortened tip suture needle; and a cutting needle.

In some configurations, the bio-dissolvable suture needle can include a tip and a base, wherein the base is configured to couple to bio-dissolvable or non-dissolvable suturing material.

In some configurations, the bio-dissolvable suture needle can include a tip and a base, and suturing material, wherein the suturing material is contiguously attached to the base.

In some configurations, the bio-dissolvable suture needle is made of at least one of: PLA, PGLA, PGLA gamma irradiated, and Polyglycolic Acid (PGA).

Use of language such as “at least one of X, Y, and Z,” “at least one of X, Y, or Z,” “at least one or more of X, Y, and Z,” “at least one or more of X, Y, or Z,” “at least one or more of X, Y, and/or Z,” or “at least one of X, Y, and/or Z,” are intended to be inclusive of both a single item (e.g., just X, or just Y, or just Z) and multiple items (e.g., {X and Y}, {X and Z}, {Y and Z}, or {X, Y, and Z}). The phrase “at least one of” and similar phrases are not intended to convey a requirement that each possible item must be present, although each possible item may be present.

The various embodiments described above are provided by way of illustration only and should not be construed to limit the scope of the disclosure. Various modifications and changes may be made to the principles described herein without following the example embodiments and applications illustrated and described herein, and without departing from the spirit and scope of the disclosure. For example, unless otherwise explicitly indicated, the steps of a process or method may be performed in an order other than the example embodiments discussed above. Likewise, unless otherwise indicated, various components may be omitted, substituted, or arranged in a configuration other than the example embodiments discussed above.

Further aspects of the present disclosure are provided by the subject matter of the following clauses.

A bio-dissolvable suturing needle, comprising: a tip; and a base.

The bio-dissolvable suturing needle of any preceding clause, wherein the base is configured to couple to bio-dissolvable or non-dissolvable suturing material.

The bio-dissolvable suturing needle of any preceding clause, further comprising: suturing material, wherein the suturing material is contiguously attached to the base, and wherein the suturing material is one of bio-dissolvable or non-dissolvable.

The bio-dissolvable suturing needle of any preceding clause, wherein the bio-dissolvable suturing needle is manufactured using 3-D printing.

The bio-dissolvable suturing needle of any preceding clause, wherein the bio-dissolvable suturing needle is manufactured using injection molds.

The bio-dissolvable suturing needle of any preceding clause, wherein the bio-dissolvable suturing needle is made of at least one of: PLA, PGLA, PGLA gamma irradiated, and Polyglycolic Acid (PGA).

The bio-dissolvable suturing needle of any preceding clause, wherein the tip comprises a trussed point.

The bio-dissolvable suturing needle of any preceding clause, wherein the tip comprises a cutting needle.

The bio-dissolvable suturing needle of any preceding clause, wherein the tip comprises a shortened tip.

The bio-dissolvable suturing needle of any preceding clause, wherein the tip comprises a regular structure.

A method of making a bio-dissolvable suture needle comprising the steps of: injecting a bio-dissolvable material into a mold having a suture needle form, resulting in at least one bio-dissolvable suture needle; and cooling the mold until the at least one bio-dissolvable suture needle solidifies.

The method of any preceding clause, the at least one bio-dissolvable suture needle comprises at a plurality of bio-dissolvable suture needles cojoined by a common base, wherein each bio-dissolvable suture needle in the plurality of bio-dissolvable suture needles can be removed from the common base prior to suturing.

The method of any preceding clause, wherein the plurality of bio-dissolvable suture needles comprises: a trussed point needle; a regular suture needle; a shortened tip suture needle; and a cutting needle.

The method of any preceding clause, wherein each needle in the at least one bio-dissolvable suture needle comprises a tip and a base, wherein the base is configured to couple to bio-dissolvable or non-dissolvable suturing material.

The method of any preceding clause, wherein each needle in the at least one bio-

dissolvable suture needle comprises a tip, a base, and suturing material, wherein the suturing material is contiguously attached to the base.

The method of any preceding clause, wherein the at least one bio-dissolvable suturing needle is manufactured using 3-D printing.

The method of any preceding clause, wherein the at least one bio-dissolvable suturing needle is manufactured using injection molds.

The method of any preceding clause, wherein the at least one bio-dissolvable suturing needle is made of at least one of: PLA, PGLA, PGLA gamma irradiated, and Polyglycolic Acid (PGA).

The method of any preceding clause, wherein the tip comprises a trussed point.

The method of any preceding clause, wherein the tip comprises a cutting needle.

The method of any preceding clause, wherein the tip comprises a shortened tip.

The method of any preceding clause, wherein the tip comprises a regular structure.

A method of making a bio-dissolvable suture needle comprising the steps of: retrieving, from a database, a three dimensional (3D) suture needle design; uploading the three dimensional suture needle design to a 3D printer; providing bio-dissolvable material to the 3D printer; and forming, via the 3D printer, the bio-dissolvable material into the bio-dissolvable suture needle according to the 3D suture needle design.

The method of claim any preceding clause, the bio-dissolvable suture needle comprising at least one of: a trussed point needle; a regular suture needle; a shortened tip suture needle; and a cutting needle.

The method of any preceding clause, wherein the bio-dissolvable suture needle comprises a tip and a base, wherein the base is configured to couple to bio-dissolvable or non-dissolvable suturing material.

The method of any preceding clause, wherein the bio-dissolvable suture needle comprises a tip and a base, and suturing material, wherein the suturing material is contiguously attached to the base.

The method of any preceding clause, wherein the bio-dissolvable suturing needle is made of at least one of: PLA, PGLA, PGLA gamma irradiated, and Polyglycolic Acid (PGA).

We claim:

1. A bio-dissolvable suturing needle, comprising:
a tip; and
a base.

2. The bio-dissolvable suturing needle of claim 1, wherein the base is configured to couple to bio-dissolvable or non-dissolvable suturing material.

3. The bio-dissolvable suturing needle of claim 1, further comprising:
suturing material, wherein the suturing material is contiguously attached to the base, and wherein the suturing material is one of bio-dissolvable or non-dissolvable.

4. The bio-dissolvable suturing needle of claim 1, wherein the bio-dissolvable suturing needle is manufactured using 3-D printing.

5. The bio-dissolvable suturing needle of claim 1, wherein the bio-dissolvable suturing needle is manufactured using injection molds.

6. The bio-dissolvable suturing needle of claim 1, wherein the bio-dissolvable suturing needle is made of at least one of: PLA, PGLA, PGLA gamma irradiated, and Polyglycolic Acid (PGA).

7. The bio-dissolvable suturing needle of claim 1, wherein the tip comprises a trussed point.

8. The bio-dissolvable suturing needle of claim 1, wherein the tip comprises a cutting needle.

9. The bio-dissolvable suturing needle of claim 1, wherein the tip comprises a shortened tip

10. The bio-dissolvable suturing needle of claim 1, wherein the tip comprises a regular structure.

11. A method of making a bio-dissolvable suture needle comprising the steps of:
injecting a bio-dissolvable material into a mold having a suture needle form, resulting in at least one bio-dissolvable suture needle; and
cooling the mold until the at least one bio-dissolvable suture needle solidifies.

12. The method of claim 11, the at least one bio-dissolvable suture needle comprises at a plurality of bio-dissolvable suture needles cojoined by a common base, wherein each bio-dissolvable suture needle in the plurality of bio-dissolvable suture needles can be removed from the common base prior to suturing.

13. The method of claim 12, wherein the plurality of bio-dissolvable suture needles comprises:
a trussed point needle;
a regular suture needle;
a shortened tip suture needle; and
a cutting needle.

14. The method of claim 11, wherein each needle in the at least one bio-dissolvable suture needle comprises a tip and a base, wherein the base is configured to couple to bio-dissolvable or non-dissolvable suturing material.

15. The method of claim 11, wherein each needle in the at least one bio-dissolvable suture needle comprises a tip, a base, and suturing material, wherein the suturing material is contiguously attached to the base.

16. A method of making a bio-dissolvable suture needle comprising the steps of:
retrieving, from a database, a three dimensional (3D) suture needle design;
uploading the three dimensional suture needle design to a 3D printer;
providing bio-dissolvable material to the 3D printer; and
forming, via the 3D printer, the bio-dissolvable material into the bio-dissolvable suture needle according to the 3D suture needle design.

17. The method of claim 16, the bio-dissolvable suture needle comprising at least one of:
a trussed point needle;
a regular suture needle;
a shortened tip suture needle; and
a cutting needle.

18. The method of claim 16, wherein the bio-dissolvable suture needle comprises a tip and a base, wherein the base is configured to couple to bio-dissolvable or non-dissolvable suturing material.

19. The method of claim 16, wherein the bio-dissolvable suture needle comprises a tip and a base, and suturing material, wherein the suturing material is contiguously attached to the base.

20. The method of claim 16, wherein the bio-dissolvable suturing needle is made of at least one of: PLA, PGLA, PGLA gamma irradiated, and Polyglycolic Acid (PGA).
